(19) United States
(12) Patent Application Publication
(10) Pub. No.: US 2003/0049284 A1
(43) Pub. Date: Mar. 13, 2003

(54) Method of increasing the solubility of alphahydrocarboxilic salts, and products obtained by the method

(76) Inventors: Jose Ruben Boccio, Buenos Aires (AR); Adrian Tomas De Paoli, Buenos Aires (AR); Eduardo Walter Ettlin, Buenos Aires (AR); Edgardo Adrian Hager, Buenos Aires (AR)

Correspondence Address: JACOBSON HOLMAN, 400 SEVENTH STREET, N.W., WASHINGTON, DC, 20004, US

(21) Appl. No.: 09/946,167

(22) Filed: Sep. 5, 2001

(51) Int. Cl. A61K 7/00; C07C 229/24; C07C 229/26

(52) U.S. Cl. 424/401; 562/579; 562/562; 562/565

(57) ABSTRACT

Method of increasing the solubility of salts of alpha hydroxy carboxylic acids, preferably used in the field of nutrition and feeding of living matter, and products obtained by the method, the products enhancing and increasing the bio-availability of macro and micro essential elements contained in the products.

### BACKGROUND OF THE INVENTION

1. Field of the Invention

The present invention relates to the field of food, nutrition, feeding and nourishing and, more particularly refers to the enhancing and increasing in the bio-availability of macro and micro essential elements for the living requirements of animals, human beings and vegetables and plants. The invention is related to the provision of better absorption by the intestine of elements that are essential to the animals and human beings.

2. Description of the Prior Art

The functional and structural organization of the living matter is based in the incorporation, into the living matter, of thirty to ninety natural elements of the Periodical Table. Most of these elements have a low atomic number but only five of these elements which are considered indispensable for the living matter in the Earth have an atomic number higher than the atomic number of Se, with Z=34, such as iodine having a Z=53 and molybdenum with a Z=42, for example. The more abundant of these elements are (in decreasing order) hydrogen, oxygen, carbon, nitrogen, which are called “organic elements” which, all together, comprises about the 99% of the cell mass, considered in terms of the percentages for the corresponding subject, relative to the total number of atoms. These elements, combined with the sulfur and phosphorous, form the macro molecules (oligomers and bio polymers) and molecular aggregates with several vital functions such as structural functions, genetic information storage function, O₂ carrying function, recognizing function, etc.

Following an importance order of the elements in the living matter, after the above “organic elements” the “macro elements” or “macro nutrients” and “micro elements” or “micro nutrients” are found. The concentration of the macro nutrients in the living matter is at least 100 mg. of the dry matter, these elements being the potassium, phosphorous, sulfur, calcium, magnesium, chlorine and sodium. The concentration of the micro nutrients in the living matter does not exceed the 100 micrograms (100g of dry matter), these elements being mainly the iron, manganese, copper, iodine, selenium, zinc. The importance of these elements varies from the animal kingdom to the vegetal kingdom, the chromium, the iodine, the zinc and the selenium have not been shown to be essential in the vegetal kingdom.

The elements are defined macro and micro nutrients based in the daily requirements, for example: 200-2000 mg. per day for the macro nutrients (Ca, Mg, K) and 0.1 to 20 mg per day for the micro nutrients (Zn, Cu, Fe, Cr, Co). Micro and macro elements are also called as a common denomination of “mineral elements” and are irreplaceable components in the tissues and body fluids at the intracellular medium, in the electrons at the Redox reactions (iron), as co-factors of several enzymes (Zn in the dehydrogenases), Mg as a co-factor in the photosynthesys and Ni in the ureases, etc.

An element is “essential” when at least one of the following conditions is satisfied:

a) it is present in the fetus or in the just born baby before any foreign contamination is detected;

b) if there is any mechanism that, by regulating the placental conveyance, protects the fetus from the excessive accumulation of the element, or the minimum quantities thereof are guaranteed at the expense of depletion;

c) if there is any homeostatic mechanism that assures the presence of the element by regulating the absorption or excretion of the element;

d) if there is a reservoir regulated by hormonal or nutritional mechanisms, or any other factor;

e) if there is a correlation between the enzymatic activity and the concentration of the involved element;

f) if it is possible to experimentally induce a deficiency in animals or plants in an extent for producing a symptomatology that can be reversed or prevented by the administration of the element. Each of the essential elements is a conditioning factor of the functional or the structural equilibrium of the individuals, and an ingesta (radicle absorption) enough to compensate the loses due to urinary and faecal disposals (in the animal kingdom) or by defoliation and fructification in the vegetal kingdom.

The following Table 1 shows the ingesta of several essential elements recommended for an adult normal individual, the values being variable with the health status, age, activity, etc.

TABLE 1

| | | |
|---|---|---|
| Calcium | 1000-2000 | mg/day |
| Magnesium | 230-350 | mg/day |
| Copper | 1.50-3.00 | mg/day |
| Iron (Fe) | 12-18 | mg/day |
| Zinc | 11-15 | mg/day |
| Manganese | 2.00-5.00 | mg/day |
| Chromium | 50-200 | μg/day |
| Molybdenum | 75-250 | μg/day |
| Nickel | 150-200 | μg/day |
| Potassium | 1.9-5.6 | g/day |

The above are average values for healthy individuals and vary with to the age and activity of the individuals.

The ingesta with essential elements, either bio-elements or nutrients, has been based, from the begining of the first 25 years in the last century, in the oral administration of salts of organic acids such as mono, di and tri-basic acid phosphates, calcium pirophosphates, or magnesium pirophosphates, or iron(II) phosphates, calcium iodate, or organic acids such as saccharate, calcium levulinate, or the calcium citrate or iron(II) citrate, etc. Further investigations have demonstrated that the bio-availability of the macro and micro nutrients depends not only of the solubility of the compounds utilized in the digestive fluids but also of the called “conveying systems” through the membranous tissues, wherein diffusion mechanisms with other tissues are combined, this mediated by specific conveying molecules.

The handling of these factors, together with the size, form, charge or polarity of the ions or molecules at the intestinal field, determines the selectivity, velocity and absorption extent, limited at 50-70% of the ingesta of a normal adult individual for Mg²+, for example.

Looking for substances that are more efficient from the diet point of view, it has been reached to the gradual substitution of the above cited traditional compounds by compounds, salts for example, of alpha hydroxy carboxylic or poly carboxylic acids, such as gluconic acids, magnesium gluconate, calcium gluconate, iron(II) gluconate, copper(II) gluconate, calcium heptonate, magnesium heptonate, calcium gluco ascorbate, or salts of dibasic organic acids, such as the iron(II) succinate or fumarate, and amino acids such as copper(II) glycinate.

Even tough the above compounds are beneficial for the living matter, such as the plants, animals and human beings, the absorption thereof by the digestive mechanisms of the individuals is a concern. It would be therefore convenient to have a product, a compound and a process to enhance and increase the absorption of such essential elements through the corresponding absorbing mechanisms of such living matter.

### SUMMARY OF THE INVENTION

It is therefore one object of the present invention to provide new means for increasing the efficiency in the bio-availability of macro and micro elements, which elements are formulated in soluble compositions that provide larger quantities of the essential elements through the intestinal tract, this absorption being in direct relationship with the conveying function through the membranes of the intestinal tissue, this being determinant of the ions and molecules flow into the inner medium, that is of the extent or quantity of the absorbed bio-elements.

It is still another object of the present invention to provide a method based in an acid-base reaction between an alpha hydroxy carboxylic acid and at least one oxide or hydroxide of the chosen bio-element, in an aqueous medium, in presence of alpha-amino acids, preferably in stoichiometric relationship. The method is based in the discovery of the inventors that the salts of the alpha hydroxy carboxylic acid, such as the gluconic acid or the lactic acid, are considerably increased in the presence of alpha amino acids or the salts thereof.

It is a further object of the present invention to provide a method of increasing the solubility of salts of alpha hydroxy carboxylic acids with at least one essential element, the method comprising the steps of:

a) reacting oxides or hydroxides of said at least one element, the oxides or hydroxides being dispersed or solubilized in an aqueous medium including an alpha hydroxy carboxylic acid or a lactone of the same in presence of quantities of an alpha amino acid, in a quantity enough to dissolve the employed base, and

b) recovering from the formed solution, by crystallization or evaporation, a product having a higher solubility, thus increasing the bio-availability of the at least one essential element as compared to the bio-availability that would be provided by the corresponding salt of the employed hydroxy carboxylic acid.

It is even another object of the present invention to provide a product to be administered to animals and human beings for feeding purposes, the product containing at least one essential element selected from zinc, copper(II), chromium(III), Co(II), Ni(II), Fe(II),K(I), Fe(III),Ca(II) and Mg(II) the product having a higher solubility and providing an increased bio-availability of the at least essential element as compared to the solubility and bio-availability of other products containing said elements, the product being obtained by reacting oxides or hydroxides of said at least one element, the oxides or hydroxides being dispersed or solubilized in an aqueous medium including an alpha hydroxy carboxylic acid or a lactone of the same in presence of quantities of an alpha amino acid, in a quantity enough to dissolve the employed base, and the product is recovered from the formed solution, by crystallization or evaporation.

It is still a further object of the invention to provide a composition for supplementing the ingesta of bio-assimilable essential elements, wherein the composition comprises a biologically acceptable vehicle and a product obtained by reacting the oxides or hydroxides of said elements dispersed and solubilized in an aqueous medium also including an alpha hydroxy carboxylic acid or a lactone thereof, in presence of an alpha amino acid in a quantity enough to dissolve the employed base.

It is a further object of the present invention to provide a food additive for increasing the content of essential macro and micro-nutrient elements which are bio-available in food and leaves fertilizers, the additive comprising, as a source of said elements, a product obtained by reacting the oxides or hydroxides of said elements dispersed and solubilized in an aqueous medium also including an alpha hydroxy carboxylic acid or a lactone thereof, in presence of an alpha amino acid in a quantity enough to dissolve the employed base and, depending on the case, recovering, by crystallization or evaporation, the product having a higher solubility and higher bio-availability of the essential element as compared to the bio-availability that would be provided by the corresponding salt of the employed hydroxy carboxylic acid, wherein combinations or associations of hydroxy carboxylic acids, such as lactic acid and gluconic acid, may be employed.

It is even a further object of the present invention to provide a use of alpha amino acids as reactant agents for increasing the solubility and bio-availability, in an aqueous medium, of the soluble, or less soluble, salts of essential elements such as calcium, magnesium, Fe(II), Fe(III), copper(II), chromium(III), Co(II), manganese(II), Zn (II) and K(I.

While references to the human beings are made in the present application, it is remarked, as it will be well known to any person skilled in the art, that all these concepts related to diet supplements of essential micro and macro elements in the man are also applicable to the agricultural and cattle activities in order to solve problems related to nutrients deficiencies and bio-availability of the herein involved elements, all of this being related to the enrichment of food and beverage rations for animals and nutrition for plants.

The above and other objects, features and advantages of this invention will be better understood when reading the following description.

### DESCRIPTION OF THE PREFERRED EMBODIMENTS

Now referring in detail to the invention a comparative analysis will be made between the solubility of certain salts of calcium of gluconic acid, or lactic acid, and said salts in presence of glicine.

### Comparative Data

In the following comparative table, compositions 2, 4 and 6 corresponds to the present invention.

1). Calcium gluconate: 30 gr/1000 ml of water at 20° C., (index Merck Ref. 1675).

2). Calcium gluconate—glicine: 300-1000 gr/1000 ml of water at 20° C. depending on the pH.

3). Fe(II) gluconate: 100 gr/1000 ml of water at 20° C. (F.C.C. IV Ed. Page 153)).

4). Fe(II) gluconate—glicine: 600 gr/1000 ml of water at 20° C.

5). Calcium lactate: 60 gr/1000 ml of water at 20° C.

6). Calcium lactate—glicine: 400 gr/1000 ml of water at 20° C.

The best mode of carrying out the invention is shown in the following examples, all the examples using reactants of pharmaceutical or analytic quality and distilled water.

### EXAMPLE 1

Solubilization of Calcium Gluconate in Presence of Glicine

127 gr. of calcium hydroxide have been dispersed in 3 liters of water under intensive stirring, at room temperature. The dispersion has been screened to eliminate insoluble clots (mesh 60-80). 258 gr. of glicine have been slowly added by maintaining the stirring and 1348 gr. of 50% gluconic acid diluted in water have been slowly added while the temperature is increased and maintained at 60-98° C. until the neutralization is reached (pH: 6.0-8.0). A clear solution is obtained by filtration, the solution containing dissolved solids 20%. 1000 gr. of a solid product that is white, odorless, light, slightly wet (it may contain about 4% humidity) hygroscopic, having a density of about 0.4-0.7 gr/ml, very soluble in water, is obtained by desiccation. Instead of the gluconic acid the corresponding lactone, previously hydrolyzed, may be employed. The obtained product is soluble in water in a 1:1 ratio at 20-25° C., forming a solution that is somewhat thick with a viscosity of about 1.2 cps. The calcium gluconate has a limited solubility of about 30-80 gr./l of water at the same temperature.

### EXAMPLE 2

Preparation of Solutions with a High Content of Fe(II)

According to the technique disclosed in Example 1, an aqueous dispersion of calcium hydroxide (Ca(H₂O) 127 gr.) in 3 liters of distilled water is prepared. Once processed like in Example 1 to eliminate the clots and insoluble particles, 258 gr. of glicine are added under stirring. A solution of 1348 gr. of gluconic acid in 1.5 liters of distilled water is added under stirring, while the temperature is increased to 60-98° C. and maintained until a clear solution and complete neutralization is reached. Once the entire neutralization (pH 6.0-8.0) is verified, 478.0 gr. of Fe(II) sulfate 7H2O dissolved in 1000 ml of water are added under stirring. The residual mass of calcium sulfate is decanted, filtered and washed and a crystalline solution is obtained containing about 15-20% of dissolved solids. The water of the solution is evaporated and a clear green, odorless and light fine powder is obtained, the powder having a density of 0.4-0.7 gr./ml, and being easily soluble in water. The obtained product has a remarkably increased solubility as compared to the Fe(II) gluconate, that is 1000 gr. of the product obtained according to the present invention, in 1 liter of water at 20-25° C., as compared to 85-100 gr. of Fe(II) gluconate under the same conditions.

### EXAMPLE 3

Reactants

| | |
|---|---|
| Zinc Oxide | 140 gr. |
| 50% Gluconic Acid | 1360 gr. |
| Glicine | 250 gr. |

Distilled Water, in a quantity enough for the 15-20% solids solution.

The method is similar to the one disclosed in Example 1, by using an enough quantity of distilled water and the final pH of the clear solution being adjusted to 5.5-7.5. After concentrating and eliminating the water from the solution, as it is indicated in Example 1, about 1000 gr. of a white product have been obtained, the product being very soluble in water containing 110 gr. of Zinc.

### EXAMPLE 4

The technique disclosed in Example 2 has been repeated by using copper sulfate instead of ferrous sulfate. In one case it has been employed:

| | |
|---|---|
| Calcium Hydroxide | 130 gr. |
| Glicine | 260 gr. |
| 50% Gluconic Acid | 1348 gr. |
| Copper sulfate 5H₂O | 250 gr. |

The dispersion of 130 gr. of calcium hydroxide in distilled water has been added with the indicated quantity of glicine, then the indicated quantity of gluconic acid at a 50% was added, diluted in 1.5 liters of water, under heating to 60-98° C. and under steady stirring.

Then the copper sulfate, previously dissolved in 1 liter of water until the entire solubilization thereof is reached, is added. After adjusting the pH between 3.5-5.5 a crystalline blue-violet solution is recovered by filtration. About 1000 gr. of a blue, light, soluble product is obtained by evaporating the water, the product containing about 105 gr. of copper.

While the mechanisms determining the important increasing of the solubility of the salts of the alpha hydroxy carboxylic acids of the above cited cations, in an aqueous medium and in presence of significant quantities of alpha amino acids, have not been determined, it is considered that the cause of such increasing in the solubility is due to the formation of a coordinating structure with the bio-elements as the central atom. In addition to this interpretation it must be taken into account that the verified increasing in the solubility may be reached by employing two or more amino acids simultaneously. Under these considerations, if the amino acid is at least one essential amino acid like the lysine, glutamic acid, etc. it will be possible to combine the solubilizing capability and the biologic effects of the essential amino acids as structural components of the proteins.

It is also possible to employ simultaneously two or more amino acids, and, in accordance to the invention, it is possible to simultaneously overcome the effects arising from the lack of essential elements and necessary amino acids. In the compositions commonly formulated for overcoming these nutritional aspects, the amino acids are employed to comply with the biologic function and, according to the invention, this is enhanced and complemented by the solubilizing function that allows the individual to have diet formulations containing higher quantities of essential bio-elements in a soluble formula.

The method of the invention may be practiced in two alternative operative ways. One of the ways implies the neutralization, in an aqueous medium, of the alpha hydroxy carboxylic acid, such as the gluconic acid or the lactic acid, in presence of a chosen amino acid, employing the oxide or the hydroxide of the chosen elements as a base and, after filtration, adjustment of pH, etc. the solution is dried.

It is also possible to add, in a gradually mode or in an alternating mode, the alpha hydroxy carboxylic acid and the amino acid in the base dispersed in an aqueous medium. Generally, it is recommended to work under a hot temperature, then cool down and complete the preparation with the conventional operations of filtering and pH adjustment, with the same alkaline hydroxides or hydroxy carboxylic acids, either with calcium hydroxide or magnesium hydroxide, for example.

As it has been previously remarked in connection to the simultaneous employment of two or more amino acids, it is also possible to employ two or more different base such as calcium hydroxide or magnesium hydroxide, for example, for neutralizing the alpha hydroxy carboxylic acid to obtain products with two different compounds: of calcium and of magnesium as essential elements. In connection to this aspect, the invention is not restricted to products having calcium and/or magnesium only, as essential elements. It is also possible to employ, for example, copper(II) bases, manganese(II) bases or chromium(III) bases. It is also possible to employ pre-formed salts of calcium, magnesium, Fe(II) and Fe(III), etc. with alpha hydroxy carboxylic acids, such as gluconic acid, for example, in partial or total substitution of the above mentioned bases, re-dissolving the same in an aqueous medium containing alpha amino acids. This alternative is also within the scope of the present invention. The inventive method provides an alternative practice based in the displacement reaction that is applicable when the bases (oxides or hydroxides) are not available. In these cases, the calcium of already prepared solutions, such as glicine calcium gluconate, is precipitated and a salt is added to the solutions, the salt having an anion capable of precipitating the calcium and the required essential element, such as copper, as copper sulfate or Fe(III) sulfate, or chromium(III) sulfate.

Before the separation of the insoluble material (CaSO₄) the solution is processed and utilized as it has been already indicated in connection to the salts obtained by neutralization. The formed solutions containing the essential element solubilized in presence of glicine, lysine, etc. can be conveniently processed by evaporation, lyophilization, etc., for obtaining more concentrated solutions or a solid residue. This may be then applied as an additive in pills or tablets, or for supplementing or fortifying foods in any event when the ingesta must be enhanced with essential elements.

While preferred embodiments of the present invention have been exemplified and described, it will be obvious to those skilled in the art that various changes and modifications may be made therein without departing from the scope of the invention as defined in the appended claims.

We claim:

1. A method of increasing the solubility of salts of alpha hydroxy carboxylic acids with at least one essential element, the method comprising the steps of:
a) reacting oxides or hydroxides of said at least one element, the oxides or hydroxides being dispersed or solubilized in an aqueous medium including an alpha hydroxy carboxylic acid or a lactone of the same in presence of quantities of an alpha amino acid, in a quantity enough to dissolve the employed base, and
b) recovering from the formed solution, by crystallization or evaporation, a product having a higher solubility, thus increasing the bio-availability of the at least one essential element as compared to the bio-availability that would be provided by the corresponding salt of the employed hydroxy carboxylic acid.

2. The method of claim 1, wherein the employed alpha-amino acid is a natural alpha-amino acid.

3. The method of claim 2, wherein the natural alpha-amino acid is selected from glicine, essential alpha-amino acid, L-lysine and L-glutamic acid.

4. The method of claim 1, wherein the employed alpha-hydroxy carboxylic acid is selected from lactic acid, poly hydroxy carboxylic acids, lactones of the poly hydroxy carboxylic acids, gluconic acid, gluconadeltagalactone, pantothenic acid.

5. The method of claim 1, wherein the essential elements are calcium, magnesium and potasium.

6. The method of claim 1, wherein the essential elements are zinc, copper(II), chromium(III), Co(II), Ni(II), Fe(II) and Fe(III).

7. The method of claim 1, wherein the solids dissolved in the reaction medium are recovered by evaporation and product is obtained as an additive for supplementing the ingesta of the essential element contained in the product.

8. The method of claim 1, wherein the solution resulting from step b), when calcium or magnesium is involved in the same, is further processed by the addition of soluble salts of another essential element selected from Fe(II), Fe(III), Ni(II), Cu(II), Co(II), all in stoichiometric quantities, wherein the calcium ions are separated by precipitation.

9. The method of claim 8, wherein the calcium ions are precipitated as calcium sulfate.

10. A composition as a supplement of bio-assimilable essential elements, wherein the composition comprises the product obtained by the method of claim 1, the composition being in combination with a biologically acceptable vehicle in a dose unit.

11. The composition of claim 10, wherein the product is solubilized in a biological acceptable vehicle selected from water, dairy product, milk, yogurt and syrup.

12. The composition of claim 10, wherein the vehicle is a soda.

13. The composition of claim 10, wherein the dose unit is selected from pellets, pills, tablets and cereals.

14. An additive for increasing the content of essential macro and micro-nutrient elements which are bio-available in food and leaves fertilizers, the additive comprising, as a source of said elements, the product obtained by the method of claim 1.

15. The additive of claim 14, wherein the essential element is selected from calcium and magnesium.

16. The additive of claim 14, wherein the alpha hydroxy carboxylic acid is selected from gluconic acid and lactic acid, and the natural alpha amino acid is selected from glicine and lysine.

17. A product to be administered to animals and human beings for feeding purposes, the product containing at least one essential element selected from zinc, copper(II), chromium(III), Co(II), Ni(II), Fe(II) and Fe(III), the product having a higher solubility and providing an increased bio-availability of the at least essential element as compared to the solubility and bio-availability of other products containing said elements, the product being obtained by reacting oxides or hydroxides of said at least one element, the oxides or hydroxides being dispersed or solubilized in an aqueous medium including an alpha hydroxy carboxylic acid or a lactone of the same in presence of quantities of an alpha amino acid, in a quantity enough to dissolve the employed base, and the product is recovered from the formed solution, by crystallization or evaporation.

18. A product to be administered to animals and human beings for feeding purposes, the product containing at least one essential element selected from zinc, copper(II), chromium(III), Co(II), Ni(II), Fe(II) and Fe(ITI), the product being obtained by the method of claim 1.

19. Use of alpha amino acids as reactant agents for increasing the solubility and bio-availability, in an aqueous medium, of the soluble salts of essential elements.

20. The use of claim 19, wherein the essential elements are selected from calcium, magnesium, Fe(II), Fe(III), copper(II), chromium(III), Co(II),manganese(II) and potassium (I).
